(19) United States
(12) Patent Application Publication
(10) Pub. No.: US 2004/0092899 A1
(43) Pub. Date: May 13, 2004

(54) Tampon and the like

(76) Inventors: James Winchester, New York, NY (US); Vadim Davankov, Moscow (RU)

Correspondence Address: Ilya Zborovsky, 6 Schoolhouse Way, Dix Hills, NY, 11746, US

(21) Appl. No.: 10/292,966

(22) Filed: Nov. 13, 2002

(51) Int. Cl. A61F 13/20

(52) U.S. Cl. 604/378

(57) ABSTRACT

A tampon and the like has a tampon body formed to be introducible into a vaginal cavity, and an adsorbing material provided in the tampon body so as to adsorb and retain toxins, etc., from a fluid which is present in a vaginal cavity.

### BACKGROUND OF THE INVENTION

The present invention relates to tampons and similar feminine products.

Catamenial tampons and similar feminine products are well known and widely used for introduction into a vaginal cavity. While the known tampons reliably perform their intended functions, they have a significant disadvantage. In particular, bacteria which are usually present in the vaginal cavity are accumulated on the tampon, multiply and cause negative consequences, and sometimes even septic shock. It is therefore believed to be clear that it is advisable to develop new tampons which avoids the disadvantages of the prior art.

### SUMMARY OF THE INVENTION

Accordingly, it is an object of the present invention to provide a tampon and the like which avoids the disadvantages of the prior art.

In keeping with these objects and with others which will become apparent hereinafter, one feature of present invention resides, briefly stated, in a tampon and the like, which has a tampon body insertable into a vaginal cavity; and an adsorbing material provided in said tampon body so as to adsorb toxins in the vaginal cavity.

When the tampon and the like is designed in accordance with the present invention, it avoids the disadvantages of the prior art. The tampon reliably adsorbs toxins and prevents their damaging action to a user.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view schematically showing a tampon in accordance with one embodiment of the present invention; and

FIG. 2 is a view showing a tampon in accordance with another embodiment of the present invention.

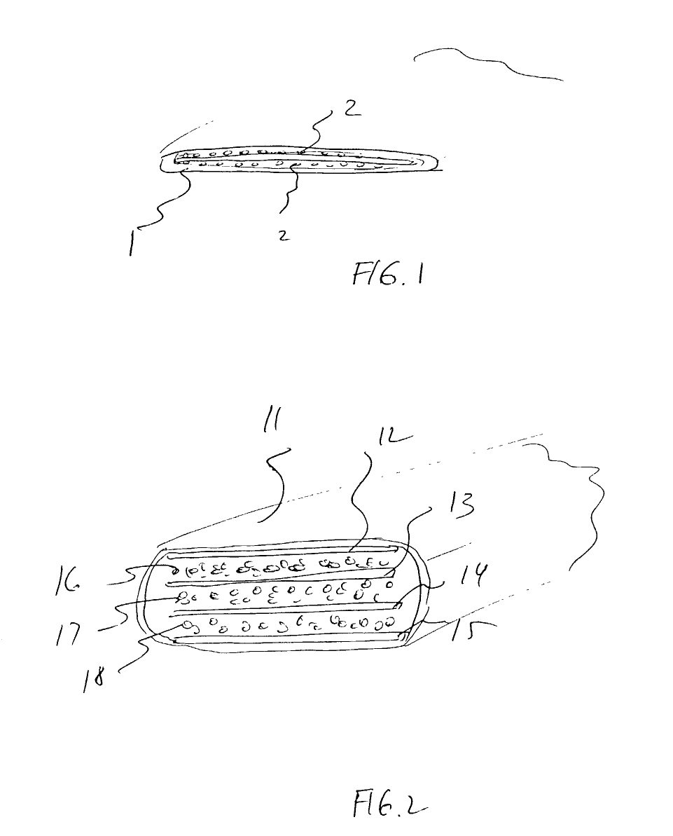

### DESCRIPTION OF PREFERRED EMBODIMENTS

A tampon in accordance with the present invention has a tampon body which is identified with reference numeral 1. The tampon body 1 can be formed as any known tampon and the tampon body is composed of a material which is suitable for this purposes. Conventionally, such a material must be substantially soft liquid absorbing, and biocompatible.

In accordance with the present invention, an additional adsorbing material 2 is provided in the tampon body 1. As can be seen from the drawings, the adsorbing material 2 is arranged close to an outer surface of the tampon body 1.

The adsorbing material 2 can be formed for example as a layer of fine-powdered porous hydrophobic polymeric adsorbent with high adsorption activity, e.g. toward enterotoxins released by bacteria (Staphylococcus Aureus), and exotoxins released by bacteria (Streptococus Groups A, B, F and G). One of the very efficient adsorbing polymers for this purpose represents a predominantly mesoporous polydivinylbenzene (or a copolymer of styrene with divinylbenzene) with additionally introduced anionic functional groups.

The particle size of the adsorbent should be less than 100 micron, preferably less that 50 micron. The polymer is not be exposed to the outer surface of the tampon and does not come into an immediate contact with the body tissue, but is positioned close to that surface. The bacterial contamination is expected to develop in the interface area between the tissue and the tampon. By placing the major part of the polymer close to the tampon surface, adsorption of toxins is facilitated.

The polymeric adsorbent 2 can be prepared in the following manner:

### EXAMPLE 1

Into a seven-liter four-necked round-bottom flask equipped with a stirrer, a thermometer and a reflux condenser, is placed the solution of 8.4 g polyvinyl alcohol-type technical grade emulsion stabilizer GM-14 in four liters of deionized water (aqueous phase). The solution of 260 ml divinylbenzene, 140 ml ethylvinylbenzene, 250 ml toluene, 250 ml n-octane and 2.94 g benzoyl peroxide (organic phase) is then added to the aqueous phase on stirring at room temperature. In 20 min, the temperature is raised to 80° C. The reaction is carried out at 80° C. for 8 hours and 90° C. for additional 2 hours. After accomplishing the copolymerization, the stabilizer is rigorously washed out with hot water (60 to 80° C.) and the above organic solvents are removed by steam distillation. The beads obtained are filtered, washed with 1 l dioxane and with deionized water. Finally, the beads are dried in oven at 60° C. overnight.

The polymer displayed apparent inner surface area of over 600 sq.m/g and total pore volume of 0.8 ml/g.

### EXAMPLE 2

As in example 1, taking 220 ml divinylbenzene, 180 ml ethylvinylbenzene, 100 ml styrene, 200 ml toluene, 200 ml n-octane and 3.0 g benzoyl peroxide as the organic phase. Inner surface area of the product obtained amounts to 600 sq.m/g.

The polymer surface is further modified as follows:

To 6 g of dry polymer prepared in Example 1, 25 ml of acetic anhydride were added, the mixture was cooled to 0° C., slowly provided with 2 ml of aqueous 40% solution of hydrogen peroxide, and stirred at 10-15° C. for 8 h. The polymer was filtered, washed with glacial acetic acid to result in epoxy groups containing material.

Then 2 g of the epoxy modified polymer, swollen with glacial acetic acid were carefully washed with dry ethyl acetate and provided with 1 ml of 2-hydroxyethyl amine in 4 ml ethyl acetate. The mixture was stirred at 40° C. for 5 h, which results in the addition of the hydroxyethylamino group to the epoxy group. The polymer was filtered, washed with water, 1 NHCl and again water to neutral pH.

In a separate experiment, diethylamine was taken instead of 2-hydroxyethyl amine. The product was washed with water, 1 N HCl and water as described above.

When the tampon is introduced into the vaginal cavity, the toxin-containing fluid inside the cavity passes to the tampon body and toxins from the fluid are adsorbed and retained in the adsorbing material.

In accordance with another embodiment of the present invention, the tampon has a tampon body which is identified as a whole with reference numeral 11 and is composed of a plurality of layers 12, 13, 14, 15, etc, each composed of a corresponding material or materials. Such a multi-layer structure is known for tampons and other feminine products. In accordance with a new inventive feature, the adsorbent material 15 also has several layers identified with reference numerals 16, 17, 18. The layers of the adsorbent material are arranged so that they are located between the layers of the tampon body 11.

When the fluid in the vaginal cavity flows to the tampon in accordance with the present invention, it passes through the multiple layers 16, 17, 18 of the adsorbing material. Thereby toxins, are reliably retained in the layers of the adsorbing material.

The tampon in accordance with the present invention eliminates the disadvantages of the prior art.

In accordance with the present invention ligands can be attached to the surface of the polymeric adsorbent and can represent antibiotics, antibodies to toxins, inhibitors of toxins. For example, they can include aliphatic alcohol.

It will be understood that each of the elements described above, or two or more together, may also find a useful application in other types of constructions differing from the types described above.

While the invention has been illustrated and described as embodied in tampon and the like, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A tampon and the like, comprising a tampon body formed to be introducible into a vaginal cavity; and an adsorbing material provided in said tampon body so as to adsorb and retain toxins, etc., from a fluid which is present in a vaginal cavity.

2. A tampon as defined in claim 1, wherein said adsorbing material is located close to an outer surface of said tampon body.

3. A tampon as defined in claim 1, wherein said tampon body is formed as a plurality of tampon body layers, said adsorbing material including a plurality of adsorbing layers, said adsorbing layers being located between said tampon body layers.

4. A tampon as defined in claim 1, wherein said adsorbing material is a fine-powdered hydrophobic polymeric adsorbent with high adsorption activity toward enterotoxins and endotoxins released by bacteria.

5. A tampon as defined in claim 4, wherein said adsorbent is a predominantly mesoporous polydivinylbenzene or a copolymer of styrene with divinylbenzene with additionally introduced anionic functional groups.

6. A tampon as defined in claim 1; and further comprising a ligand attached to a surface of powder particles of said polymeric adsorbent

7. A tampon as defined in claim 6, wherein said ligand is an element selected from the group consisting of an antibiotic, a toxin antibody, and a toxin inhibitor.
